(19) United States
(12) Patent Application Publication
(10) Pub. No.: US 2001/0028878 A1
(43) Pub. Date: Oct. 11, 2001

(54) In vitro maturation of human oocytes in one day

(76) Inventors: Svend Lindenberg, Skodsborg (DK); Anne Lis Mikkelsen, Frederiksberg (DK); Steven Dale Smith, Bronshoj (DK); Kjell Bertheussen, Eidkjosen (NO)

Correspondence Address: BIRCH STEWART KOLASCH & BIRCH, PO BOX 747, FALLS CHURCH, VA, 22040-0747, US

(21) Appl. No.: 09/746,656

(22) Filed: Dec. 22, 2000

Related U.S. Application Data
(63) Continuation-in-part of application No. PCT/DK99/00345, filed on Jun. 22, 1999.
(60) Provisional application No. 60/090,115, filed on Jun. 22, 1998.

(30) Foreign Application Priority Data
Jun. 22, 1998 (DK) PA 1998 00885

(51) Int. Cl. A01K 67/027

(52) U.S. Cl. 424/93.7; 800/21

(57) ABSTRACT

The present invention relates to a method for in vitro maturation of a human oocyte by culturing an immature human oocyte in a cell culture medium for 10-30 hours. The maturation end point is metaphase II.

### BACKGROUND OF THE INVENTION

The normal ovulating woman will recruit approx. 300 immature oocytes for each menstrual cycle. This recruitment takes place before the actual cycle. At the day of menstruation, around 20-30 immature oocytes will still be present. Normally, during a process of apoptosis all but one oocyte will die before ovulation. At day 5-10 of the menstrual cycle, approx. 10-15 immature oocytes will be present in their small follicles being 5-12 mm in diameter. Some still growing and some starting to undergo an apoptotic process.

Conventional in vitro fertilisation (IVF), treatment for special cases of severe male and female infertility, is based on retrieval of mature human oocytes followed by fertilisation of the mature oocytes with spermatozoa. The recruitment of human mature oocytes is accomplished through several complicated forms of hormone treatments, often with discomfort or risk for the woman involved. These hormone treatments will especially be a problem in the future, as IVF is increasingly offered to perfectly normal women in these programs due to their husband's poor sperm quality. Because of the risk, discomfort and cost of the hormonal stimulation several other approaches have been tried during the years. One of the most simple ways to avoid hormonal stimulation has been not to stimulate with hormones at all. In animals in vitro maturation (IVM) has become an efficient method for producing oocytes for fertilisation, but until now recorded success rates for clinical human IVM have been low (Cha, Trounson, Barnes, Russell). This treatment regimen, however, requires in vitro maturation of the oocytes.

### DETAILED DESCRIPTION OF THE INVENTION

The present invention relates to a method for in vitro maturation of a human oocyte by culturing an immature human oocyte for 10-30 hours in a cell culture medium. It is possible to obtain immature human oocytes from women in infertility treatment by aspirating and extracting these oocytes from ovarian tissue. Furthermore, if a patient is diagnosed with cancer, ovarian tissue can be dissected out and frozen prior to initiation of treatment that might cause sterility as cytostatic or radiation treatment often does. Immature oocytes can later be extracted from the frozen ovarian tissue. These immature oocytes can then be finally matured.

The method of the present invention will preferably start with immature or not fully matured oocytes. In the woman the oocytes will be recognised as oocytes with a tight cumulus mass, no polar bodies or Germinal vesicles visible. These oocytes are readily recognised by a person involved in routine IVF-treatments as being immature oocytes.

The advantages of starting with immature oocytes as described above are several. A woman in treatment for infertility normally undergoes a complicated hormonal treatment for many days for gaining a sufficient number of mature prophase II oocytes for in vitro fertilisation. This hormonal treatment encompasses pain, discomfort, stress, and risk of ovarian hyperstimulation syndrome, a condition feared among patients and doctors. This hormonal therapy is instituted to rescue immature oocytes which will otherwise undergo apoptosis. Thus, these hormones are essential for allowing the immature oocytes to mature within the ovary in the substantial number needed for IVF. If no hormones are administrated only one oocyte will mature as seen in normal ovulating women. By releasing or removing these immature oocytes from the ovary prior to initiation of the apoptotic processes and mature them further in vitro, the woman can avoid the risk and discomfort associated with hormonal treatment and still have a sufficient number of mature Metaphase II (MF-II) oocytes for subsequent fertilisation (e.g. standard IVF treatment).

Oocyte maturation is the final stage of oocyte development that prepares for fertilisation and embryo development. It can be divided into two general processes: nuclear maturation and cytoplasmic maturation. Nuclear maturation is defined as the resumption of meiosis and progression to MF-II while cytoplasmic maturation is defined as the extragenomic changes that prepare the egg for activation, pronuclear formation, and early embryogenesis. Thus, by an immature female oocyte is understood an ova that upon contact with a mature sperm cell will not complete the mitotic division and accept the genetic material from the sperm cell and form a fertilised cell. In one embodiment of the invention, the non-fertilisable, i.e. immature, female ova or oocyte, is a meiotic cell that is in a stage prior to germinal vesicle break-down (GVB), entrance into MF-I, and the follicle is antral or pre-antral.

By MF-II is understood an oocyte with 1 polar body, expanded cumulus complex and which has finally gone through a germinal vesicle break-down. These oocytes are readily recognised by a routine technician normally handling oocytes for IVF.

In example 1, it is illustrated that decreasing the culture time from 36 h to 26 h causes no quantitative difference on the subsequent fertilization, cleavage and embryo score. However, as illustrated in example 2, the implantation rate and the development of clinical pregnancies is not affected by culturing the oocytes for 28 hours. Thus, it is preferred to culture the immature oocytes for less than 30 hours, such as less than 29 hours, less than 28 hours, less than 27 hours, less than 26 hours, less than 25 hours, less than 24 hours, or even less than 23 hours. However, as stated above, there is a minimum limit for how short the culture time can be in order for the maturation of the cumulus cells and the nuclear maturation to take place. The minimum culture time for the immature oocytes is 10 hours, such as 11 hours, 12 hours, such as 13 hours, such as 14 hours, such as 15 hours, such as 16 hours, such as 17 hours, such as 18 hours, such as 19 hours, or even 20 hours.

Without being bound by the present theory, we anticipate that the number of oocytes maturing to MF-II will be increasing over time. Thus, at 28 hours 73% of the oocytes are at MF-II, at 36 hours 77% of the oocytes are at MF-II. At later time-points, the percentage of oocytes in MF-II is increased. Prior to 28 h a lower percentage of oocytes will be in MF-II, however, at a very early stage, no oocytes will have had the time to mature. However, among the higher percentage obtain at e.g. 36 h, 48 h or even 56 h, most of the oocytes have been in MF-II arrest for 20-30 h, which places them well past the optimal fertilization time and may compromise their developmental competence. It is thus expected that this arrest decreases the capability of the oocyte to, when fertilized, implant.

Shortening the maturation period from 36 hours to 28 hours did not significantly reduce the yield of matured oocytes or affected the subsequent developmental capacity of the oocytes in our study. However, in a preferred embodiment of the present invention, the developmental capacity, expressed as the implantation rate, is increased.

In addition, the 28-hour IVM period had a significant benefit in that it allowed the inseminations to be performed during working hours; they had to be performed at night when the 36-hour IVM schedule was used.

The oocytes are matured in a medium. If hormones or serum derived substances are to be added to the medium, recombinant hormones or serum derived substances are preferred. Preliminary results indicate that it has no effect on pregnancy rates to lower the content of Human Serum Albumin (HSA) in the medium from 5% to 0.5%. Thus, in one embodiment of the present invention, the contents of HSA, Bovine Serum Albumin (BSA) or other directly serum derived product is less than 0.5%, such as 0.4%, 0.3%, 0.2%, 0.1%, e.g. less than 0.05% and even less than 0.01%. In an alternative embodiment, the culture medium contains BSA or HSA obtained by recombinant methods, thereby eliminating the inter-mammal serum contact. In one embodiment the immature human oocytes are cultured in a chemically defined medium without addition of directly serum-derived products or the patients' own serum or any other serum product derived directly from a mammal, such as a human or cattle.

The advantage of using a medium without biologically extracted serum substances is that the risk of transferring viruses or other pathogen or harmful particles to the medium and subsequently to the embryo is substantially reduced or non-existing. Furthermore, serum probably contains a factor, presently unknown, that inhibits the synchronised maturation of the nucleus, cytoplasma and cumulus expansion.

Thus, one aspect of the present invention relates to a method to avoid infection or contamination of a non-fertilisable oocyte with known and/or unknown infectious agents (such as prions, viroids, virus, mycoplasma, bacteria, fungi) during in vitro maturation of the non-fertilisable oocyte, by culturing the oocyte in a medium without components originating from sources at least potentially containing infectious agents. In a preferred embodiment of that aspect, the method relates to avoiding contamination with toxic, teratogenic, carcinogenic, or mutagenic components.

The basic culture medium should be one that can both support the oocyte as well as its cumulus cells. It is well known in the art that addition of gonadotropins and/or steroid such as E₂ to the maturation medium enhances the fertilizability and/or developmental ability of e.g. cattle, monkey, and human oocytes. The addition of the gonadotrophins (FSH and hCG) to human IVM medium has been widely used but their optimal concentrations (or absolute necessity) have not been fully characterised. The cumulus cells can be considered a type of co-culture and as with other types of somatic cells, they generally require moderately high protein levels in the medium. It has been suggested that oocytes need to be primed with oestrogen in order to develop Ca++ oscillations. The medium of the present invention thus preferably contains oestrogens in concentrations of 0.1 to 10 μg/mL estradiol 17-β, e.g. 0.3 to 3 μg/mL estradiol 17-β, preferably 1 μg/mL estradiol 17-β.

In one embodiment of the present invention, the medium among other factors contains ATA (Aurin Tricarboxylic Acid) as an anti-apoptotic agent. The advantage of ATA is that it might provide optimal conditions to inhibit apoptotic processes otherwise deteriorating the oocyte maturation. Another advantage of the presence of ATA is that it allows the concentration of serum derived products, such as HSA or BSA to be lowered, such that the concentration of the serum derived products is zero.

In the present invention the term “apoptosis” should be understood as a controlled cell death, where the cell itself destroys its nuclear DNA, envisioned by DNA stand-breaks. The usage of an anti-apoptotic agent is preferred due to the fact that the oocyte retrieved is already engaged in an apoptotic process in the cumulus mass. When apoptosis starts in the oocyte-cumulus complex, this will signal the start of maturation. However, as this process progresses in the normal ovary, it will induce apoptosis in the oocyte. By removing the oocyte from the ovary after initiation of the apoptotic signal, which induces start of maturation, full development will take place in the medium with e.g. ATA to stop further apoptosis.

The medium could be a medium as described in PCT/EP97/06721 hereby incorporated by reference. As an additive to the medium, a preferred additive is Medi-Cult SSR 4x, Medi-Cult SSR 4xa, Medi-Cult SSR 4xb, Medi-Cult SSR1 or Medi-Cult SSR2. As the basic medium, the preferred medium is Medi-Cult BBEM as described in Example 1. Insulin is a component of the above mentioned media. However, recent research has pointed out, that the presence of insulin in the culture medium has a negative effect on the chance for successful pregnancy. Therefore, in a preferred embodiment of the present invention, the culture medium is a culture medium as described above without insulin.

The culture of the immature oocytes for 10-30 hours to MF-II is preferably followed by fertilisation and pregnancy after implantation into the female. In one embodiment of the invention, the pregnancy rate obtained when oocytes matured for 10-30 hours in vitro is more than 10%, such as more than 13%, 15%, 18%, 20%, 21%, 22%, 23%, 24%, 25%, 26%, 27%, 28%, 29%, 30%, 31%, 32%, 33%, 34%, 35% or even more than 40%.

Apart from the contents of the medium, other factors are important in achieving this goal. These factors include the timing of the oocyte aspiration and the size of the follicles by the time of aspiration.

In a presently preferred embodiment of the present invention, the oocyte is derived from ovarian follicles with a diameter of 8-12 mm. The advantage of such small follicles is that they are present in substantial numbers without severe hormonally treatment, they can be seen by ultrasound and an ultrasonically guided transvaginal puncture of the follicles is possible to perform in order to retrieve the oocyte.

An early apoptotic phase or an artificial plateau phase in the follicular growth may mimic the final preovulatory follicular maturation terms of developmental competence.

In vitro maturation of human oocytes is not only related to growth of the follicle, but also to the size of the follicles and the oocytes. The human oocyte appears to have a size dependant ability to resume meiosis and complete maturation. A decreased maturation rate and cleavage rate of oocytes obtained from follicles <8 mm is observed. Results suggest that capacity of human oocyte maturation is closely correlated with follicular maturation. As mentioned above, the maturing oocytes retrieved are in an early apoptotic phase. Thus, with increasing size of the oocytes the risk of obtaining oocytes in a late apoptotic phase, that is close to dead cells, increases. Based on these experiences, the preferred size of the oocytes retrieved is less than 12 mm.

In a preferred embodiment of the present invention the following steps are followed:

retrieval of ovarian follicles with a diameter of 8-12 mm by transvaginally ultrasound guided aspiration

culturing the retrieved ovarian oocytes (in prophase) as described above to synchronise cumulus-, cytoplasm-, and nuclear maturations up to MF-II.

### REFERENCES

Cha K Y, Koo J J, Ko J J, Choi D H, Han S Y, Yoon T K. Pregnancy after in vitro fertilization of human follicular oocytes collected from nonstimulated cycles, their culture in vitro and their transfer in a donor oocyte program. Fertil Steril 1991;55:109-13

Russell J B, Knezevich K M, Fabian K, Dickson J A: Unstimulated immature oocyte retrieval: early versus midfollicular endometrial priming. Fertil Steril 1997;67:616-20.

Barnes F L, Kausche A K, Tiglias J, Wood C, Wilton L, Trounson A. Production of embryos from in vitro-matured primary oocytes. Fertil Steril 1996;65:1151-6.

Trounson A, Wood C, Kaunsche A. In vitro maturation and the fertilization and developmental competance of oocytes recovered from untreated polycystic ovarian patients. Fertil Steril 1994;62:353-62.

Cha K Y, Chung H M, Han S Y, Yoon T K, Oum K B, Chung M K. Successful in vitro maturation, fertilization and pregnancy by using immature follicular oocytes collected from unstimulated polycystic ovarian syndrome patients. Fertil Steril Abstract O-044;1996:Supl.S23.

Mikkelsen A L ,Smith S D, Lindenberg S. In vitro maturation of human oocytes from regularly menstruating women may be successful without FSH priming. Hum. Reprod. 1998;14:1847-51

### EXAMPLES

It is to be understood that the examples described below are illustrative of embodiments of the present invention, and the invention is not intended to be so limited.

### Example 1

Culturing of Immature Oocytes

Immature oocytes are aspirated transvaginally with a 17 g Cook needle (Cook, Australia) under low aspiration pressure. Follicular aspirates are collected into tubes or syringes containing warmed Flushing Medium (Medi-Cult, Denmark).

All manipulations are carried out at 37° C. Follicular aspirates are filtered (Falcon 1060) to remove erythrocytes and small cellular debris. The retained cells are resuspended in equilibrated BBEM (Medi-Cult, Denmark) with bicarbonate and HEPEs buffers and then oocytes are isolated under a stereomicroscope and washed twice in BBEM (pH 7.2-7.4, mosmol/kg: 285±8, Modified EBS with lactate, MEM non essential amino acids, 2.5 mM HEPES, 0.1 mM Taurine, 200 mM Ultra-glutamine, 0.2 mM sodium pyruvate, 0.5 mM d-glucose, 0.8 mM MgSo4 anhydrous, 3.6 mM Ca-lactate, 1 mM NaH2PO4, 5.4 mM K2SO4, 110 mM NaCl, 1 ml/l SSR4xb (Medi-Cult, Denmark)). Immature oocytes are incubated in BBEM in 5% CO₂ and air at 37° C. for 2 h before being transferred into IVM medium. The IVM medium used for oocytes from patients 1-3 consists of BBEM supplemented with SSR4x 1:1000 (Medi-Cult, Denmark), 0.075 IU/mL recombinant human FSH, 0.5 IU/mL hCG (both from Serono, Denmark), 1 μg/mL estradiol 17-β (Sigma, Denmark), and 5% HSA (Statens Serum Institut, Denmark). Oocytes form patient 1, were cultured in an IVM medium containing only 0.5% HSA.

The IVM medium used for oocytes from patients A-C consists of Tissue Culture Medium 199 (Sigma), 0.075 IU/mL recombinant human FSH, 0.5 IU/mL hCG (both from Serono, Denmark), 1 μg/mL estradiol 17-β (Sigma, Denmark), and 5% HSA.

Oocytes are cultured singly in 25 μL drops of IVM medium under paraffin oil at 37° C. in 5% CO₂ and humidified air. During the growth cumulus expansion is observed as a sign of healthy maturing oocytes. Images are recorded at approximately 24 h and again at either 36 or 48 h of culture, and the number of cells in MF-II are counted.

Oocytes are denuded with hyaluronidase (IVF Science, Sweden) and mechanical pipetting. Motile sperm are prepared by Puresperm™ (Cryos, Denmark) gradient separation or by swim-up. For ICSI denuded oocytes are placed individually into 5 μL drops of sperm prep medium (Medi-Cult, Denmark) and 2 μL of sperm suspension is placed into a 10 μL drop of PVP (IVF Science, Sweden). All metaphase II oocytes are inseminated by ICSI and then placed into 10 μL drops of BBEM and cultured in 5% CO₂ and humidified air at 37° C. Approximately 10-20 h after insemination oocytes are examined at 300× for the presence of pronuclei as a measure of successful fertilization. Embryos are cultured to day 2 or 3 (day 0=day of insemination) at which time suitable embryos (maximum of 2) are replaced into the women. Suitable embryos are those that are cleaved. The suitable embryos are scored on a scale from 1 (best) to 4 (worst) prior to replacement.

Oocytes from patient (pt) 1-3 are cultured for 36h. Oocytes from pt A-C are cultured for 26 h.

TABLE 1

| | | Cumulus | | | | |
|---|---|---|---|---|---|---|
| Pt. | No of Oocytes | expansion | MF-II | Fertilized | Cleaved | Score |
| 1 | 5 | 0 | 2 | 2 | 2 | 2.1 |
| 2 | 3 | 3 | 2 | 1 | 1 | 2.2 |
| 3 | 3 | 3 | 3 | 2 | 2 | 2.1 |
| A | 1 | 1 | 1 | 1 | 1 | 3.0 |
| B | 2 | 2 | 2 | 2 | 2 | 2.1 |
| C | 8 | 6 | 6 | 4 | 3 | 2.1 |

These results as presented in Table 1 show that there is no quantitative difference between the culture for 26 h and the culture for 36 h.

### Example 2

Contents of Medi-Cult Media

TABLE 2
SSR1 contents

| Compound | 1000 × per liter | |
|---|---|---|
| Trisodium citrate-dihydrate | 7.35 | g |
| Citric acid | 3.15 | g |
| Pluronic F-68 | 20 | g |
| Aurintricarboxylic acid (ATA) | 1.27 | g |
| Ethylenediaminetetraacetic acid Fe(III)-Na-chelate | 1.20 | g |
| dihydrate | | |
| EDTA-Na2 (triplex III) | 0.372 | g |
| Trace elements (100 000×) | | |
| EDTA-Na2; | 5.211 | g |
| Trisodium citrate-dihydrate | 0.294 | g |
| Zinc sulfate-heptahydrate | 2.875 | g |
| Copper(II)sulfate-pentahydrate | 0.499 | g |
| Manganese sulfate, H₂O | 0.017 | ml |
| Nikkel(II)-nitrat-hexahydrat | 0.0058 | g |
| Ammoniumaluminiumsulfate-12-hydrate | 0.092 | g |
| Potassium-chromium sulfate-pentahydrate | 0.05 | g |
| Cobalt(II)-chloride-hexahydrate | 0.048 | g |
| Selenium dioxide | 0.111 | g |

TABLE 3
SSR2 contents

| Compound (1000×) | pr liter | |
|---|---|---|
| Trisodium citrate-dihydrate | 7.35 | g |
| Citric acid | 3.15 | g |
| Pluronic F-68 | 20 | g |
| Aurintricarboxylic acid (ATA) | 1.27 | g |
| Ethylenediaminetetraacetic acid Fe(III)-Na-chelate | 1.20 | g |
| dihydrate | | |
| EDTA-Na2 (triplex III) | 0.372 | g |
| HCl, 1N, | 5 | g |
| Human insulin recombinant (NOVO) | 0.50 | g |
| Trace elements (100 000×) | | |
| EDTA-Na2; | 5.211 | g |
| Trisodium citrate-dihydrate | 0.294 | g |
| Zinc sulfate-heptahydrate | 2.875 | g |
| Copper(II)sulfate-pentahydrate | 0.499 | g |
| Manganese sulfate, H₂O | 0.017 | ml |
| Nickel(II)-nitrate-hexahydrate | 0.0058 | g |
| Ammoniumaluminiumsulfate-12-hydrate | 0.092 | g |
| Potassium-chromium sulfate-pentahydrate | 0.05 | g |
| Cobalt(II)-chloride-hexahydrate | 0.048 | g |
| Selenium dioxide | 0.111 | g |

TABLE 4
SSR4x contents

| Compound (1000×) | pr liter | |
|---|---|---|
| Trisodium citrate-dihydrate | 7.35 | g |
| Citric acid | 3.15 | g |
| Pluronic F-68 | 20 | g |
| Aurintricarboxylic acid (ATA) | 1.27 | g |
| Ethylenediaminetetraacetic acid Fe(III)-Na-chelate | 1.20 | g |
| dihydrate | | |
| EDTA-Na2 (triplex III) | 0.372 | g |
| HCl, 1N, | 5 | g |
| Human insulin recombinant (NOVO) | 0.50 | g |
| Ethanol | 1000 | ml |
| Cholesterol | 2.0 | g |
| Polyvinyl pyrrolidone (PVP 10) | 250 | g |
| Acetic acid (glacial) 100% | 6.0 | ml |
| Ethanolamine | 1.2 | ml |
| Trace elements (100 000×) | | |
| EDTA-Na2; | 5.211 | g |
| Trisodium citrate-dihydrate | 0.294 | g |
| Zinc sulfate-heptahydrate | 2.875 | g |
| Copper(II)sulfate-pentahydrate | 0.499 | g |
| Manganese sulfate, H₂O | 0.017 | ml |
| Nickel(II)-nitrate-hexahydrate | 0.0058 | g |
| Ammoniumaluminiumsulfate-12-hydrate | 0.092 | g |
| Potassium-chromium sulfate-pentahydrate | 0.05 | g |
| Cobalt(II)-chloride-hexahydrate | 0.048 | g |
| Selenium dioxide | 0.111 | g |

TABLE 5
SSR4xa contents

| Compound (1000×) | pr liter | |
|---|---|---|
| Trisodium citrate-dihydrate | 7.35 | g |
| Citric acid | 3.15 | g |
| Pluronic F-68 | 20 | g |
| Aurintricarboxylic acid (ATA) | 1.27 | g |
| Ethylenediaminetetraacetic acid Fe(III)-Na-chelate | 1.20 | g |
| dihydrate | | |
| EDTA-Na2 (triplex III) | 0.372 | g |
| HCl, 1N, | 5 | g |
| Human insulin recombinant (NOVO) | 0.50 | g |
| Ethanol | 1000 | ml |
| Polyvinyl pyrrolidone (PVP 10) | 250 | g |
| Acetic acid (glacial) 100% | 6.0 | ml |
| Ethanolamine | 1.2 | ml |
| Trace elements (100 000×) | | |
| EDTA-Na2; | 5.211 | g |
| Trisodium citrate-dihydrate | 0.294 | g |
| Zinc sulfate-heptahydrate | 2.875 | g |
| Copper(II)sulfate-pentahydrate | 0.499 | g |
| Manganese sulfate, H₂O | 0.017 | ml |
| Nickel(II)-nitrate-hexahydrate | 0.0058 | g |
| Ammoniumaluminiumsulfate-12-hydrate | 0.092 | g |
| Potassium-chromium sulfate-pentahydrate | 0.05 | g |
| Cobalt(II)-chloride-hexahydrate | 0.048 | g |
| Selenium dioxide | 0.111 | g |

TABLE 6
SSR4xb contents

| Compound (1000×) | pr liter | |
|---|---|---|
| Trisodium citrate-dihydrate | 7.35 | g |
| Citric acid | 3.15 | g |
| Pluronic F-68 | 20 | g |
| Aurintricarboxylic acid (ATA) | 1.27 | g |
| Ethylenediaminetetraacetic acid Fe(III)-Na-chelate | 1.20 | g |
| dihydrate | | |
| EDTA-Na2 (triplex III) | 0.372 | g |
| HCl, 1N, | 5 | g |
| Human insulin recombinant (NOVO) | 0.50 | g |
| Ethanol | 1000 | ml |
| Cholesteryl acetate | 2.0 | g |
| Polyvinyl pyrrolidone (PVP 10) | 250 | g |
| Acetic acid (glacial) 100% | 6.0 | ml |
| Ethanolamine | 1.2 | ml |
| Trace elements (100 000×) | | |
| EDTA-Na2; | 5.211 | g |
| Trisodium citrate-dihydrate | 0.294 | g |
| Zinc sulfate-heptahydrate | 2.875 | g |
| Copper(II)sulfate-pentahydrate | 0.499 | g |
| Manganese sulfate, H₂O | 0.017 | ml |
| Nickel(II)-nitrate-hexahydrate | 0.0058 | g |
| Ammoniumaluminiumsulfate-12-hydrate | 0.092 | g |
| Potassium-chromium sulfate-pentahydrate | 0.05 | g |
| Cobalt(II)-chloride-hexahydrate | 0.048 | g |
| Selenium dioxide | 0.111 | g |

TABLE 7
SSR3 contents

| Compound (1000×) | pr liter | |
|---|---|---|
| Trisodium citrate-dihydrate | 7.35 | g |
| Citric acid | 3.15 | g |
| Pluronic F-68 | 20 | g |
| Aurintricarboxylic acid (ATA) | 1.27 | g |
| Ethylenediaminetetraacetic acid Fe(III)-Na-chelate | 1.20 | g |
| dihydrate | | |
| EDTA-Na2 (triplex III) | 0.372 | g |
| HCl, 1N, | 5 | g |
| Human insulin recombinant (NOVO) | 0.50 | g |
| Ethanolamine | 1.2 | ml |
| Trace elements (100 000×) | | |
| EDTA-Na2; | 5.211 | g |
| Trisodium citrate-dihydrate | 0.294 | g |
| Zinc sulfate-heptahydrate | 2.875 | g |
| Copper(II)sulfate-pentahydrate | 0.499 | g |
| Manganese sulfate, H₂O | 0.017 | ml |
| Nickel(II)-nitrate-hexahydrate | 0.0058 | g |
| Ammoniumaluminiumsulfate-12-hydrate | 0.092 | g |
| Potassium-chromium sulfate-pentahydrate | 0.05 | g |
| Cobalt(II)-chloride-hexahydrate | 0.048 | g |
| Selenium dioxide | 0.111 | g |

### Example 3

Development of Human Oocytes Matured in vitro for 28 or 36 Hours

The patient group consisted of 48 women with regular menstrual cycles who were scheduled for ICSI or IVF treatment because of male factor infertility and/or tubular disease. None of the women had polycystic ovaries or anovulation. All the women gave their informed consent. The protocols used were approved by the central and local institutional review boards. The clinical treatment protocol did not include FSH priming and was described in detail elsewhere (Mikkelsen).

In brief, follicle development on the ovaries was monitored by transvaginal ultrasonography beginning on cycle day 3, and oocytes were aspirated between cycle days 8 and 12 after the leading follicle had reached 10 mm in diameter. Endometrial priming with 2 mg of 17β-E₂ given orally tid was begun on the day of oocyte aspiration. Two days after oocyte aspiration, treatment with intravaginal progesterone suppositories (200 mg tid) was initiated. Both treatments were continued until cycle day 14, when the serum hCG level was measured. If the test result was positive, the treatments were continued until 50 days of gestation.

Oocyte Recovery, Maturation, and Insemination, and Embryo Culture

Immature oocytes were aspirated transvaginally with an ultrasound-guided needle (Trounson), and the aspirates were collected in tubes containing prewarmed Ham's F-10 medium with heparin (Life Technologies, Roedovre, Denmark). All laboratory manipulations were performed at 37° C. Follicular aspirates were filtered (Falcon 1060; Life Technologies) to remove erythrocytes and small cellular debris. The retained cells then were resuspended in equilibrated Ham's F-10 medium that contained both bicarbonate and HEPES buffers and was supplemented with 2 mg/mL of human serum albumin (Statens Serum Institut, Copenhagen, Denmark).

The oocytes were isolated under a stereomicroscope and washed twice in the same medium, where they were held for 2-4 hours. The oocytes and/or their cumulus investments were classified as follows: multilayered cumulus, sparse cumulus, nude, or atretic. Only oocytes that were classified as having a multilayered or sparse cumulus were used for the experiments.

The IVM medium consisted of Tissue Culture Medium 199 (Sigma, Rødovre, Denmark) supplemented with 0.075 IU/mL of recombinant human FSH (Serono, Geneva, Switzerland), 0.5 IU/mL of hCG (Serono), 1 μg/mL of 17β-E₂ (Sigma), 0.30 mM of sodium pyruvate (Sigma), 1,500 IU/mL of penicillin G (Sigma), 50 mg/mL of streptomycin sulfate (Sigma), and 10% patient serum. The oocytes were cultured singly in 25-μL drops of IVM medium under paraffin oil at 37° C. in 5% CO₂ and humidified air.

Images were recorded at ×300 at the start of IVM, at 22-23 hours of IVM, and at the end of IVM (28 or 36 hours from the start of aspiration), both before and after the oocytes were prepared for ICSI. All patients' oocytes were inseminated by ICSI to confirm microscopically the presence of the first polar body, which was the criterion used to classify oocytes as matured to metaphase II.

The oocytes were denuded with hyaluronidase (IVF Science, Gothenburg, Sweden) and mechanical pipetting. Motile sperm were prepared by either Puresperm (Cryos, Copenhagen, Denmark) gradient separation or the swim-up technique. For ICSI, denuded oocytes were placed individually into 5 μL drops of Sperm Prep Medium (Medicult, Copenhagen, Denmark), and 2 μL of sperm suspension was placed into a 10 μL drop of polyvinylpyrrolidone (IVF Science).

All metaphase II oocytes were inseminated by ICSI and then cultured individually in 10 μL droplets of IVF medium (Medi-Cult) in Falcon petri dishes (Life Technologies) under oil in 5% CO₂ and humidified air at 37° C. The day after insemination, the oocytes were examined for the presence of pronuclei. Embryos were cultured to day 2.5 or day 3 (day 0=day of insemination), at which time suitable embryos (<2) were replaced into the uterus. No assisted hatching was performed.

Experimental Groups

In group 1, the oocytes were inseminated 28 hours after the start of aspiration. The mean patient age was 31 years (range, 25-38 years). Twenty-seven patients underwent a total of 29 cycles.

In group 2, the oocytes were inseminated 36 hours after the start of aspiration. The mean patient age was 32 years (range, 25-37 years). Twenty-one patients underwent a total of 26 cycles.

Statistical Analysis

Statistical analysis was performed using the Student's t-test. p<0.05 was considered statistically significant.

TABLE 8
Maturation and development of oocytes matured in vitro for 28 hours
(group 1) or 36 hours (group 2)

| Variable | Group 1* | Group 2* |
|---|---|---|
| No. of cycles | 29 | 26 |
| No. of oocytes cultured (%) | 107(100) | 84(100) |
| No. of metaphase II oocytes (%) | 78(73) | 65(77) |
| No. of metaphase II oocytes fertilized (%) | 56(72) | 51(78) |
| No. of metaphase II oocytes cleaved (%) | 50(64) | 49(75) |
| No. of cycles with ET | 21 | 20 |
| No. of embryos transferred | 37 | 33 |
| No. of implantations (fetal heartbeats) | 4 | 4 |
| No. of clinical pregnancies (%) | 4(14)# | 4(15) |

*There were no statistically significant differences between groups.
#One pregnancy ended in an abortion at < 12 weeks of gestation.

The maximum size of the leading follicle on the day of oocyte aspiration was 10-14 mm, and the subordinate follicles characteristically were 5-8 mm. The rate of oocyte retrieval from these follicles was estimated at 50%-70%.

In group 1, a total of 172 ova were recovered from 29 aspiration sessions, of these, 107 cumulus-enclosed oocytes were used in the experiments. In group 2, 26 aspiration sessions yielded a total of 136 ova; of these, 84 cumulus enclosed oocytes were used in the experiments.

In group 1, microscopic observation of cumulus-oocyte complexes at 22-23 hours after the start of maturation revealed that 26 (24%) of 107 had a visible first polar body. Similar rates of maturation to metaphase II were observed for oocytes that underwent 28 hours or 36 hours of maturation before insemination (73% and 77%, respectively). After insemination, 72% of the injected ova were fertilized in group 1 compared with 78% in group 2. The cleavage rates of injected ova were 64% and 75% in groups 1 and 2, respectively (Table 8). None of the differences were statistically significant. The mean (±SEM) cell counts on the day of transfer were 5.08±1.4 and 4.27±1.4 for groups 1 and 2, respectively. The embryos in group 1 were cultured for 3 days, whereas those in group 2 were cultured for 2.5 days. This may account for the difference in cell numbers. Eight patients in group 1 did not undergo ET because of failure of maturation or fertilization or because of abnormal fertilization. The 21 patients who underwent ET received a mean of 1.76 embryos. Four patients became clinically pregnant. One had a spontaneous abortion at <12 weeks of gestation. The other pregnancies gave birth to healthy infants.

Six patients in group 2 did not undergo ET because of the recovery of only atretic oocytes at aspiration, lack of maturation, fertilization failure, or poor embryo quality at the time of transfer. In the 20 patients who underwent ET, a mean of 1.65 embryos were replaced. Four patients became clinically pregnant and all subsequently gave birth to healthy infants.

Our findings suggest that a substantial proportion of oocytes reach metaphase II soon after the onset at 20 hours, even without the use of gonadotropin priming. This is borne out by the results of IVM for 28 hours; after denuding, 73% of all oocytes were at metaphase II. At 36 hours of IVM, 77% of the ova were at metaphase II, this is comparable to the 69% reported by Barnes et al. (Barnes) and suggests that the major burst of maturation has passed by this point.

1. A method for in vitro maturation and fertilization of a human oocyte characterized by the steps of:
(a) culturing an immature human oocyte in a cell culture medium for 10-30 hours, followed by fertilization.

2. A method according to claim 1 wherein the immature oocyte is in the prophase.

3. A method according to claim 1, further comprising the step of:
(b) transferring the fertilized oocyte of step (a) to the uterus.

4. A method according to claim 1, wherein the oocyte is cultured for a period of 20-30 hours before fertilization.

5. A method according to claim 1, wherein the oocyte is cultured for a period of 20-29 hours before fertilization.

6. A method according to claim 1, wherein the oocyte is cultured for a period of 20-28 hours before fertilization.

7. A method according to claim 1, wherein the immature oocyte is obtained from a women who has not received hormonal stimulation prior to aspiration in the menstrual cycle wherein the oocytes are aspirated.
